(19) United States
(12) Patent Application Publication
(10) Pub. No.: US 2020/0104782 A1
(43) Pub. Date: Apr. 2, 2020

(54) ARTIFICIAL INTELLIGENCE AUTOMATED PLANNING BASED ON BIOMEDICAL PARAMETERS

(71) Applicant: INTERNATIONAL BUSINESS MACHINES CORPORATION, Armonk, NY (US)

(72) Inventors: Michael Katz, Elmsford, NY (US); Dany Moshkovich, Yokneam Ilit (IL); Alexander Tuisov, Migdal HaEmek (IL)

(21) Appl. No.: 16/144,028

(22) Filed: Sep. 27, 2018

(51) Int. Cl. G06Q 10/06; G16H 10/60; G06F 9/54

(52) U.S. Cl. CPC G06Q 10/063116; G06F 9/54; G16H 10/60

(57) ABSTRACT

A method for developing an automated scheduling application includes receiving, by a computer, a user's schedule including a plurality of activities, each activity in the plurality of activities is associated with one or more constraints, associating biomedical parameters of the user with execution of the plurality of activities to determine an effect over time of each activity on the biomedical parameters, the associating is performed using a first application programming interface, based on the association of the biomedical parameters with the execution of the plurality of activities, identifying one or more evaluators including a predefined evaluation criteria to calculate a score for the user's schedule, the determining is performed using a second application programming interface, and performing a local search heuristic to modify and optimize the user's schedule, where the local search heuristic is performed using a third application programing interface.

### BACKGROUND

The present invention generally relates to the field of artificial intelligence, and more particularly to a method, system and computer program product for automated planning and scheduling of activities affecting biomedical and well-being parameters of a user.

Automated planning and scheduling, sometimes denoted as AI planning, is a branch of artificial intelligence that involves the realization of strategies or action sequences. Automated planning can be summarized as: given an initial state, a desired goal, and a set of possible actions, finding an optimal sequence of actions which leads from the initial state to a state satisfying the goal. Automated planning is often a complex task, involving solutions that have to be discovered and optimized in multidimensional space. Solutions usually resort to iterative trial and error processes that can be implemented in artificial intelligence. Accordingly, developing applications and business solutions to perform automated planning often requires scientific expertise in first order logic-based languages, and deep understanding of the algorithms that are used to solve such models.

### SUMMARY

The following embodiments and aspects thereof are described and illustrated in conjunction with systems, tools and methods which are meant to be exemplary and illustrative, not limiting in scope.

In accordance with one aspect of the present invention, a system for developing an automated scheduling application includes at least one hardware processor; and a non-transitory computer-readable storage medium having stored thereon program instructions, the program instructions executable by the at least one hardware processor to: provide a schedule generator programmed to permit generating an initial schedule including a plurality of activities, where said plurality of activities are governed by a plurality of constraints; provide a first application programming interface (API) programmed to permit associating a plurality of biomedical parameters of a user with the execution by the user of said activities; provide a second API programmed to permit defining a plurality of evaluators based, at least in part, on said biomedical parameters and said execution of said activities, where said evaluators are used for calculating a score for the initial schedule; and provide a third API programmed to apply a local search heuristic, to: (i) generate a candidate schedule by modifying the initial schedule based on a set of allowed modifications, where said allowed modifications are determined, at least in part, based on said constraints and said evaluators, (ii) calculate a score for the candidate schedule based on said evaluators, (iii) select one of the candidate schedule and the initial schedule as a current schedule, based upon the highest of the score of the candidate schedule and the score of the initial schedule, (iv) repeat steps (i)-(iii) until said score of the current schedule does not increase, (v) restart said local search heuristic based on a restart strategy selected from a set of restart strategies, and (vi) repeat steps (i)-(v) until said score of the current schedule does not increase, whereby the final current schedule is designated as a final schedule.

In accordance with another aspect of the present invention, a software development kit for developing an automated scheduling application, the software development kit including program code embodied on a non-transitory computer-readable storage medium, the program code executable by at least one hardware processor to: provide a schedule generator programmed to permit generating an initial schedule including a plurality of activities, where said plurality of activities are governed by a plurality of constraints; provide a first application programming interface (API) programmed to permit associating a plurality of biomedical parameters of a user with the execution by the user of said activities; provide a second API programmed to permit defining a plurality of evaluators based, at least in part, on said biomedical parameters and said execution of said activities, where said evaluators are used for calculating a score for the initial schedule; and provide a third API programmed to apply a local search heuristic, to: (i) generate a candidate schedule by modifying the initial schedule based on a set of allowed modifications, where said allowed modifications are determined, at least in part, based on said constraints and said evaluators, (ii) calculate a score for the candidate schedule based on said evaluators, (iii) select one of the candidate schedule and the initial schedule as a current schedule, based upon the highest of the score of the candidate schedule and the score of the initial schedule, (iv) repeat steps (i)-(iii) until said score of the current schedule does not increase, (v) restart said local search heuristic based on a restart strategy selected from a set of restart strategies, and (vi) repeat steps (i)-(v) until said score of the current schedule does not increase, whereby the final current schedule is designated as a final schedule.

In some embodiments, the constraints are selected from the group consisting of: user availability, activity duration range, a temporal precedence among two or more activities, a relationship among two or more activities, an importance attribute associated with an activity, whether an activity is mandatory or optional, and a numerical or quantitative value associated with an activity.

In some embodiments, the biomedical parameters consist of at least one of (a) a library of predefined biomedical parameters, and (b) user-defined biomedical parameters.

In some embodiments, the biomedical parameters are selected from the group consisting of: heart rate, blood pressure, blood sugar level, number of calories consumed, number of carbohydrates consumed, number of calories burned, and type and quantity of medication taken by the user.

In some embodiments, the evaluators are selected from the group consisting of: time spent outside a predefined safe zone for a given biomedical parameter, drug dosage consumed by the user, deviation from a scheduled activity, and deviation from a medical guideline.

In some embodiments, the restart strategies are selected from the group consisting of: restart from a historical schedule, restart based on a user-defined schedule, restart based on a randomly-selected schedule, and restart based on a schedule obtained by abstracting problem aspects.

In some embodiments, the allowed modifications consist of at least one of (a) a library of predefined allowed modifications and (b) user-defined allowed modifications.

In some embodiments, the allowed modifications are selected from the group consisting of: changing a start time of an activity, changing a duration of an activity, adding an activity, removing an activity, changing a temporal ordering of two or more activities, applying a predefined time offset to one or more activities, and increasing or reducing a quantity associated with an activity.

In some embodiments, the instructions further include continuously monitoring an execution of said final schedule by the user, where said monitoring is based, at least in part, on at least one of (a) the execution of said activities by the user, and (b) biomedical parameters of the user. In some embodiments, the instructions further include: calculating a new score for the final schedule based at least in part on said monitoring, and if said new score violates a predetermined condition, repeating steps (i)-(vi).

In addition to the exemplary aspects and embodiments described above, further aspects and embodiments will become apparent by reference to the figures and by study of the following detailed description.

### BRIEF DESCRIPTION OF THE DRAWINGS

The following detailed description, given by way of example and not intended to limit the invention solely thereto, will best be appreciated in conjunction with the accompanying drawings, in which:

FIG. 1 is a block diagram of a software development kit for software developers, according to an embodiment of the present disclosure;

FIGS. 2A-2E show an exemplary mobile application to create a schedule plan for a user in the healthcare domain, according to an embodiment of the present disclosure;

FIG. 3 is a functional block diagram of an application to create a schedule plan for a user in the healthcare domain, according to an embodiment of the present disclosure;

FIG. 4 is a simplified flowchart of the operational steps carried out by the application to create the schedule plan for a user in the healthcare domain, according to an embodiment of the present disclosure;

FIGS. 5 is a block diagram of internal and external components of computers and servers, according to an embodiment of the present disclosure;

FIG. 6 is a block diagram of an illustrative cloud computing environment, according to an embodiment of the present disclosure; and

FIG. 7 is a block diagram of functional layers of the illustrative cloud computing environment of FIG. 6, according to an embodiment of the present disclosure.

The drawings are not necessarily to scale. The drawings are merely schematic representations, not intended to portray specific parameters of the invention. The drawings are intended to depict only typical embodiments of the invention. In the drawings, like numbering represents like elements.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Typically, humans plan their everyday activities in order to decrease medical risks or improve their health condition. This is particularly true for people with chronic health conditions, such as diabetes or high blood pressure. Such planning is often a complex task, involving multiple effects and constraints that must be considered. Tools that allow supporting planning of daily activities are of great importance in the healthcare industry. Development of such tools is a complex task that requires scientific expertise in first order logic-based languages (such as PDDL) and deep understanding of the algorithms that are used to solve such models on top of domain expertise. These issues are prohibiting the use of planning methods in mobile business solutions by the general software developer community. For instance, currently there is a large body of work on general AI task planning. These works model and solve generic planning problems, disregarding specific aspect of the healthcare domain. Further, these methods typically focus on modeling and solving spatio-temporal computational problems and are not intended for software developers.

Embodiments of the present disclosure generally relate to the field of artificial intelligence, and more particularly to a method, system and computer program product for automated planning and scheduling of activities affecting biomedical and well-being parameters of a user. Specifically, the following described exemplary embodiments provide a method, system, and computer program product to, among other things, model and solve planning problems using object-oriented programming. Therefore, the present embodiment has the capacity to improve the technical field of artificial intelligence by, at a minimum, enabling easy integration of automated domain-specific modeling and planning tools into (mobile) applications and business solutions, without the need for specialized knowledge and skills in artificial intelligence by software/application developers, thus improving developer's productivity.

More specifically, embodiments of the present disclosure improve developer's productivity by providing tools including developer-oriented libraries and application programming interfaces (APIs) which enable developers to easily integrate modules for modeling and solving scheduling problems into applications and business solutions in the healthcare space. For example, modeling and solving scheduling problems may utilize Java-based and representation state transfer (REST)-based developer-oriented libraries and APIs to enable simplified integration into mobile business solutions. According to an embodiment, an object-oriented programming approach may be utilized to model and solve automated (AI) planning problems, since object-oriented programming may allow developers of applications to create native and intuitive problem models.

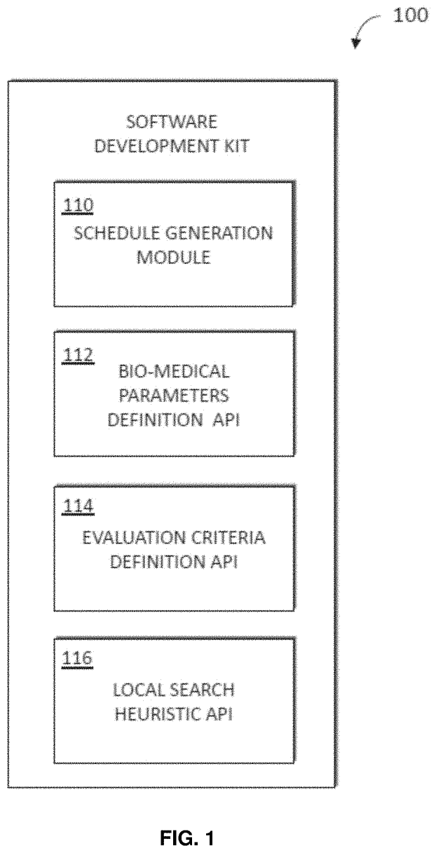

Referring now to FIG. 1, a software development kit 100 including one or more tools, such as APIs and libraries, which application developers may integrate into a schedule planning application is shown, according to an embodiment of the present disclosure. In this embodiment, a schedule generation module 110 may enable developers to define and model a multi-dimensional space of parameters for generating schedule components and their possible modifications. Such parameters may include, for example, activity types, temporal aspects of the activities, a set of constraints that must not be violated by a solution schedule, as well as other requirements and preferences.

A biomedical parameters definition API 112 may allow software or application developers (hereinafter referred to as “developers”) to define and associate biomedical parameters of a user with the execution by the user of a schedule of activities, thereby encoding the effect, over time, of these activities on the user's biomedical parameters and other well-being criteria. For example, developers can incorporate and take account of the effects, over time, of each activity on a predefined set of biomedical measurements of a user, such as heart rate, blood pressure, blood sugar levels, and the like. Additional biomedical parameters (such as calories burned) may be defined by the developer and incorporated through activity-specific evaluators. The activity-specific evaluators (hereinafter referred to as “evaluators”), include a predefined set of criteria to evaluate the quality, importance, amount, or value of a physical activity on biomedical parameters. For example, the amount of burned calories can be derived from the type and duration of a physical activity (e.g., running), based on known human medical constants.

An evaluation criteria definition API 114 may enable the developers to define a schedule evaluation criteria (evaluators), as well as means for their weighted aggregation. Such criteria may include a predefined evaluation criteria, such as time spent outside a predefined safe range for a given biomedical measurement, consumed drug dosages, deviation from original schedule, deviation from medical guidelines, etc. In addition, the developer is able to define and incorporate additional evaluation criteria.

Based on the defined space of schedule parameters and evaluators, a planner API, such as a local search heuristic API 116, may then be employed to modify and optimize an initial schedule within a defined set of allowed modifications. It should be noted that an optimized plan is one which satisfies the evaluation criteria previously defined. Additionally, the described software development kit 100 may allow monitoring the execution of the optimized plan, and dynamic, real-time optimization adjustments to the optimized plan as it is being executed.

The local search heuristic API 116 may include a heuristic search-based planning system, such as a local search algorithm. In many optimization problems, such as schedule planning, it is less important how the goal is reached, so long as it satisfies an evaluation criteria. In such cases, a local search strategy can be used to find optimized solutions to the planning problem. Local search is an iterative algorithm that begins with a user-determined or randomly chosen initial state. A “neighborhood” of solutions is defined and built for an initial solution. The local search then picks a subsequent solution from the universe of neighboring solutions, and compares it to the initial solution based on one or more criteria. If the subsequent solution better satisfies the evaluation criteria compared to the initial solution, it is deemed to be the ‘current solution’ within the system, and another local search is performed, until the criteria is maximized, or a time or iteration limit is reached.

In some embodiments, a local search heuristic which searches for an optimum solution is used. In such embodiments, neighboring solutions are determined based on the allowed modifications space. The allowed modifications space can be determined using predefined default modifications and/or user-defined modifications. In some embodiments, the local search is configured to employ restarts. For example, if no improving configurations are present in the neighborhood of solutions, the local search may become stuck at a locally optimal or maximal point. Typically, a local-optima problem can be cured by using restarts, i.e., repeated local searches with different initial conditions.

The software development kit 100 may also allow for adjusting the modification space to a specific problem at hand, e.g., changing the start time of the event by a predefined time offset, increasing or reducing the amount of carbohydrates, adding or removing a certain activity, etc. For example, the software development kit 100 allows modifying a schedule plan by changing a single activity or a collection of activities, using either predefined default modifications or user-defined modifications. In some embodiments, the software development kit 100 enforces a limit on the time of the search process, to facilitate its online usage.

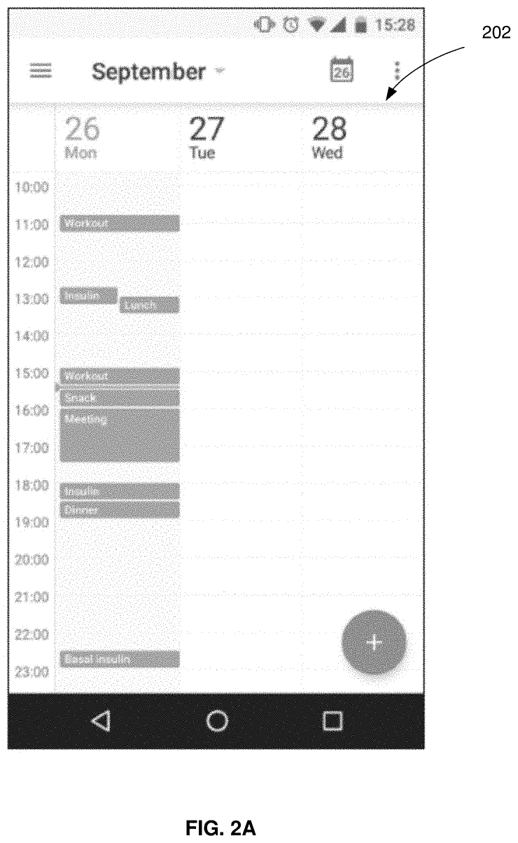

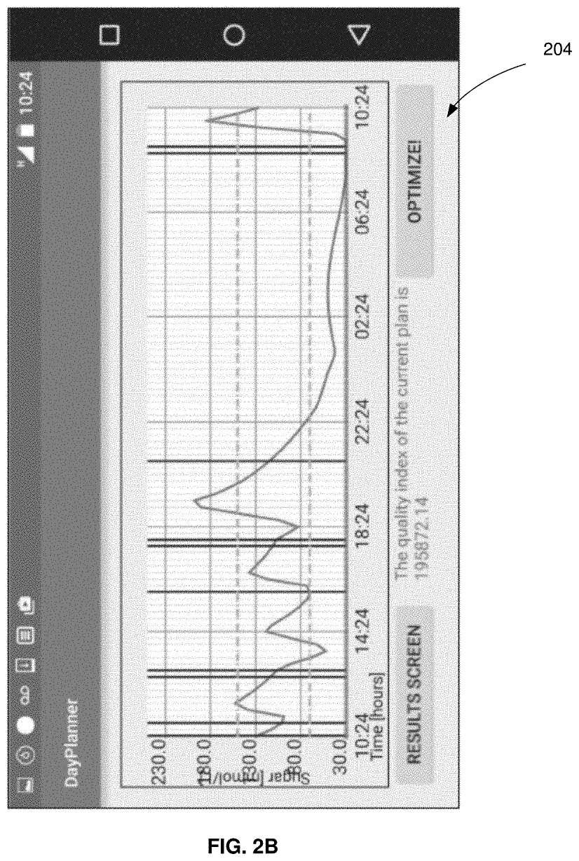

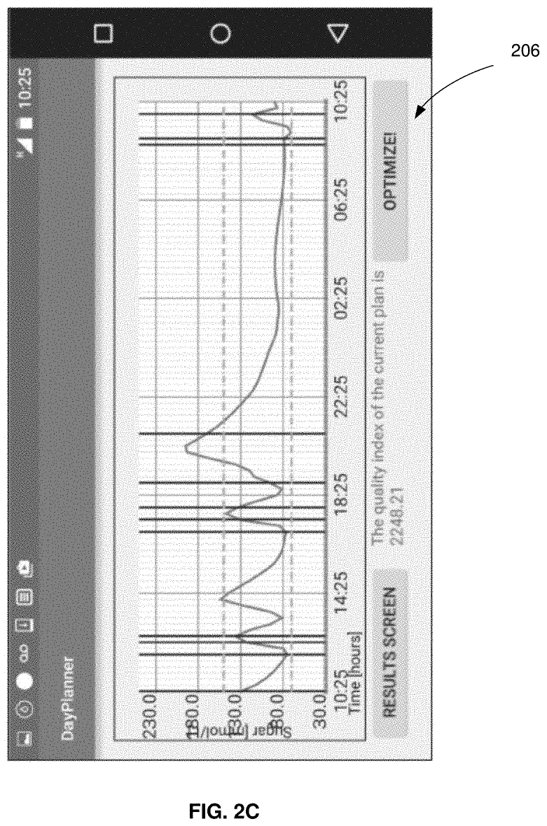

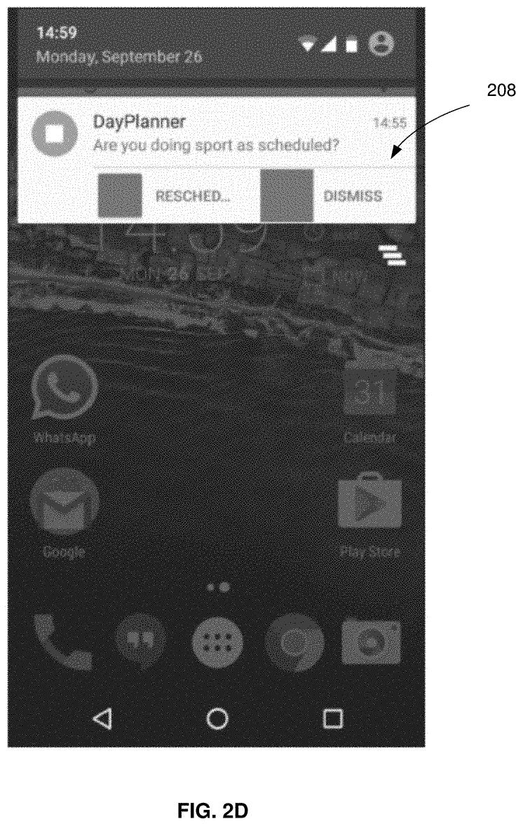

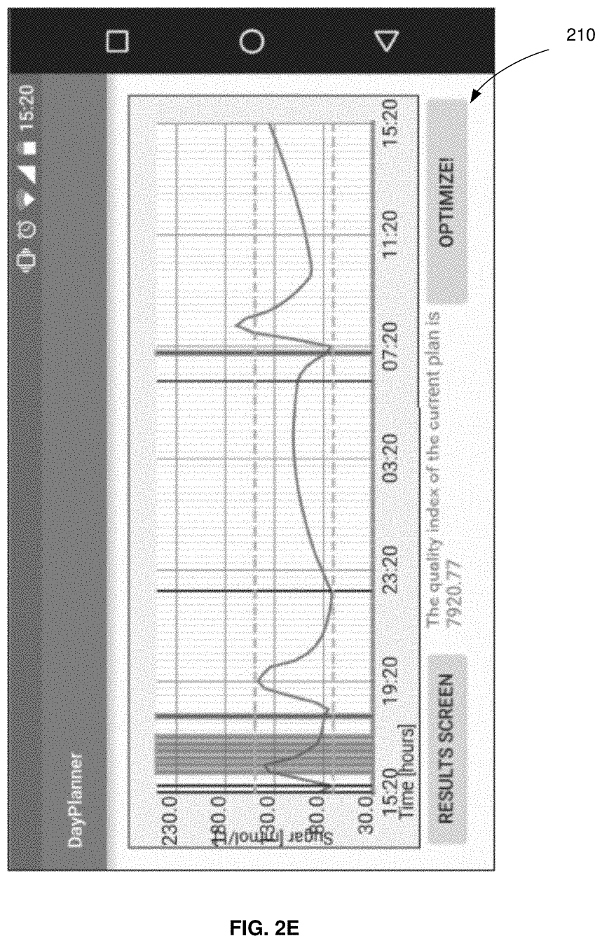

Referring now to FIGS. 2A-2E, screenshots from an exemplary application for planning a schedule for a user are shown, according to an embodiment of the present disclosure. The exemplary application is intended to generate, optimize and monitor the execution of a schedule plan for a user suffering from diabetes. The application may be developed using the software development kit 100 described in FIG. 1, and incorporate elements from the API libraries described in FIG. 1. The application may be capable of running on a mobile device, such as a telephone or a tablet computer

FIG. 2A is a screenshot of an exemplary graphic user-interface 202 to facilitate the creation of a user-defined schedule of activities. In some embodiments, the user may select from among a list of predefined default schedules available in the application.

FIG. 2B depicts a plan results screen 204 including a visual representation (in the form of a graph) of expected blood sugar levels throughout the day, based on the initial planned schedule of activities. In the figure, activities are represented by vertical lines. In some embodiments, the application evaluates a quality index of the initial plan based on one or more evaluation criteria, including biomedical parameters such as blood sugar levels.

FIG. 2C shows the results screen after optimization 206 of the initial plan, based on automatic modifications implemented by the application to satisfy the evaluation criteria. For example, the application may modify an initial plan by adding an activity, changing the start time of an activity, and/or increasing or reducing the duration of another value associated with an activity.

After modifying and optimizing the initial plan, the application may monitor on an ongoing basis the execution of the plan. For example, as shown in FIG. 2D, the application prompts (via window 208) a user to report on the progress of one or more activities included in the plan. In some embodiments, the application may allow the user to manually modify a parameter of the plan, e.g., reschedule a planned activity. In other embodiments, the application may receive real-time input regarding one or more biomedical parameters of the user, including, but not limited to, blood sugar levels, heart rate, blood pressure, and/or calories burned. User's biomedical input may, for example, be reported by the user or otherwise received directly from one or more devices communicatively connected to the application. Based on the ongoing monitoring of the execution of the plan, user's input, and additional inputs, the application dynamically modifies in real-time any current plan and updates the iterative optimization process described above. FIG. 2E shows an updated results screen 210 after the dynamic optimization, based on, for example, user's input or user-initiated modifications.

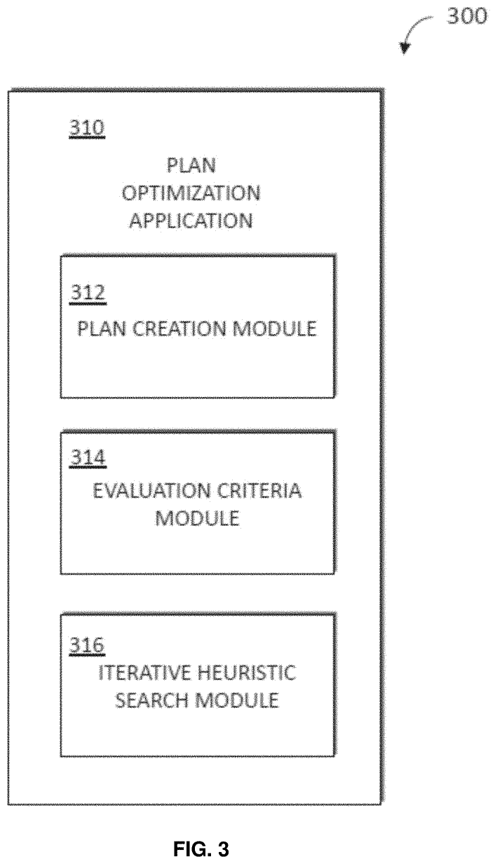

Referring now to FIG. 3, a functional block diagram 300 of an application to create a schedule plan for a user in the healthcare domain is shown, according to an embodiment of the present disclosure. A plan optimization application 310 may include a plan creation module 312, an evaluation criteria module 314, and iterative heuristic search module 316, which may be used by the plan optimization application 310 to model and solve schedule planning problems relating in the healthcare domain.

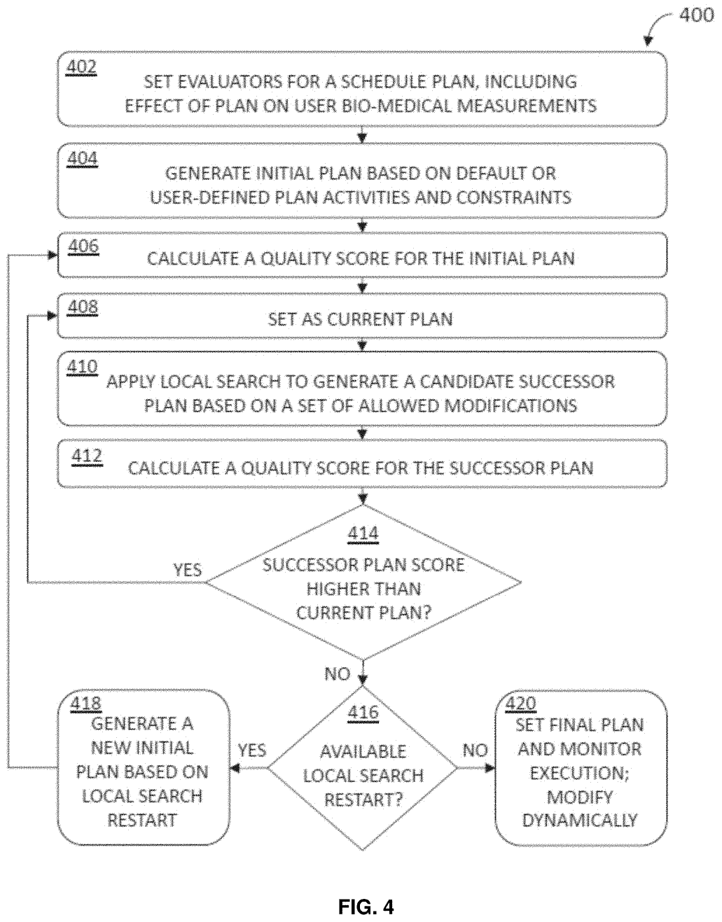

Referring now to FIG. 4, a flowchart 400 of the operational steps carried out by the application to create the schedule for a user within the healthcare domain is shown, according to an embodiment of the present disclosure. In some embodiments, the application (e.g., the plan optimization application 310 of FIG. 3) includes a mobile application running on a mobile computing device (for example, a telephone, a tablet computer, a laptop computer, etc.). In other embodiments, the application may be running on a cloud infrastructure and is accessible by a user from various user devices through an interface, such as a web browser or a local software client.

At 402, one or more evaluation criteria are defined for evaluating a plan, based on biomedical and other related well-being parameters of a user.

At 404, an initial (e.g., daily) plan or schedule of activities for the user is created within the application, using a scheduling tool (plan creation module 312 in FIG. 3) The initial schedule is stored as a digital data file including data associated with a list of activities. The scheduling tool also provides for predefined and/or user-defined constrains for various activities. The initial schedule allows defining constraints on each activity in the schedule, such as slot availability, duration, and ordering (before, after, or at the same time). In addition, the initial schedule enables to define potential values or other attributes for each activity, such as type with corresponding range and precision for numerical values. The initial schedule may also enable defining the importance of each activity (e.g., “optional,” “mandatory”). For example, it is possible to specify that an insulin injection activity “must” be performed from 30 min to 10 min before the lunch activity, as well as a potential amount of carbohydrates to be consumed during lunch.

At 406, the effects of the various scheduled activities on the user's biomedical parameters (e.g., heart rate, blood pressure, blood sugar levels, etc.) are evaluated (for example, by the evaluation criteria module of FIG. 3). Then, a quality index or score is calculated for the initial plan, based on the evaluation. In some embodiments, the application (or program) may use predefined evaluators. Specifically, the application may also provide a library of predefined evaluators, such as time spent outside predefined safe zone for a given biomedical parameter, drug dosage consumed, deviation from original schedule, deviation from medical guidelines, etc.

At 408, the initial plan is set as the current plan. At 410, an optimization process of the current plan begins by performing an iterative local search. A candidate successor plan is generated from a universe of “neighboring” plans determined by the allowed modifications space. At 412, a quality score is calculated for the candidate successor plan.

At 414, the selected best successor plan is compared against the initial plan, based on the quality score assigned to each of the plans in the comparison. If the successor plan has a better score than the initial plan, the process may iteratively repeat steps 408-414, where the successor is set as the current plan, a new candidate successor plan is generated and compared to the current plan. This process continues until a quality score of the current plan is maximized (i.e., does not improve). In some embodiments, the allowed modification space can be adjusted to a specific problem at hand such as, for example, changing the start time of the event by a predefined time offset, increasing or reducing the amount of carbohydrates, adding or removing a certain activity, etc. For example, allowed modifications can be included to a scheduled plan by modifying a single activity or a collection of activities, using either predefined default modifications or user-defined modifications. In some embodiments, a limit is enforced on the time of the search process, to enable its online usage.

At 416, when the quality score is maximized (i.e., no better solution is found within the neighboring universe of solutions), a local search restart is performed (e.g., by the iterative heuristic search module 316 of FIG. 3) to redefine the neighboring universe. The local search restart may be chosen from several search restart options from which a new initial plan is generated at 418. Steps 408-414 are then repeated for the new initial plan as described above, until a quality score for the restarted search does not improve, where a new restart may be selected at 416 again.

As described above, local search restarts may be employed in cases where no improving configurations are present in the neighborhood of solutions, and the local search may become stuck at a locally optimal or maximal point. A local-optima problem can be cured by using restarts, i.e., repeated local searches with different initial conditions. In some embodiments, the present program is configured to employ random and heuristic-based restarts, such as restarts from historical schedules, whereby a new initial schedule is selected from previously-considered schedules in the user's history. Another potential restart may be based on a user-defined schedule, e.g., a schedule manually defined by a user, possibly by manually rescheduling, adding, and/or removing activities. A restart may also be based on a randomly-selected schedule, e.g., randomly-selected times for activities within the predefined time windows. Finally, a restart may be based on a schedule obtained by abstracting problem aspects, where the program abstracts away some restrictions that make a schedule valid, such as predefined time period between specified activities, etc.

Once all restarts have been exhausted the iterative process ceases, and a best-scoring plan is set as the final plan at 420 and presented to the user. The application may then monitor the execution of the plan, based, e.g., on user reports and other inputs. The application may continuously search for optimal solutions based on dynamic evaluations of the current plan.

As noted above, in some embodiments, the present disclosure may employ one or more heuristic algorithms for modeling and planning a schedule. One of such algorithms may be a local search algorithm configured to modify an initial schedule plan by searching neighboring solutions based on an allowed modification space. The following is an exemplary local search algorithm which may be employed by the application, according to an embodiment of the present disclosure:

```
LocalSearch(construct_initial_schedule( ))
LocalSearch(init)
  current := init
  best := evaluate(current)
  while true
    succ := get_best_successor(find_successors(current))
    if best < evaluate(succ)
      return LocalSearch(heuristic_restart(current))
    current := succ
    best := evaluate(current)
find_successors(schedule)
  successors := { }
  for modification in AllAllowedModifications
    succ := applyModification(schedule, modification)
    if isValidSchedule(succ)
      successors := successors U {succ}
  return successors
heuristic_restart(schedule)
  return one of (based on schedule):
    random_walk_restart(depth, schedule)
    one_activity_modification(parameter, schedule)
    user_defined_restart(schedule)
```

The previously described embodiments provide a method, system, and program product for modeling and solving problems related to planning human activities that affect biomedical parameters. Embodiments of the present disclosure, provide Java-based developer-oriented libraries and APIs for planning user activities targeting medical aspects in order to enable easy integration of automated activity planning into mobile business solutions thereby improving productivity of mobile developers.

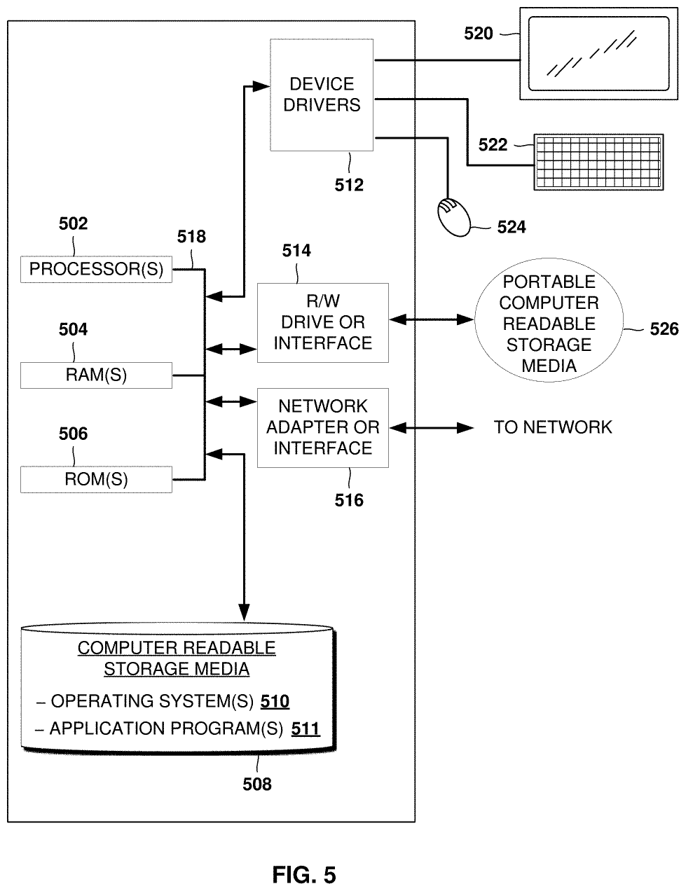

Referring now to FIG. 5, a block diagram 500 of internal and external components of a generic computer system in which embodiments of the present disclosure can be implemented is shown according to an embodiment of the present disclosure. It should be appreciated that FIG. 5 provides only an illustration of one implementation and does not imply any limitations with regard to the environments in which different embodiments may be implemented. Many modifications to the depicted environments may be made based on design and implementation requirements.

The generic computer system of FIG. 5 may include one or more processors 502, one or more computer-readable RAMs 504, one or more computer-readable ROMs 506, one or more computer readable storage media 508, device drivers 512, read/write drive or interface 514, network adapter or interface 516, all interconnected over a communications fabric 518. Communications fabric 518 may be implemented with any architecture designed for passing data and/or control information between processors (such as microprocessors, communications and network processors, etc.), system memory, peripheral devices, and any other hardware components within a system.

One or more operating systems 510, and one or more application programs 511, for example, the plan optimization application 310 (FIG. 3), are stored on one or more of the computer readable storage media 508 for execution by one or more of the processors 502 via one or more of the respective RAMs 404 (which typically include cache memory). In the illustrated embodiment, each of the computer readable storage media 508 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

The generic computer system may also include a R/W drive or interface 514 to read from and write to one or more portable computer readable storage media 526. Application programs 511 may be stored on one or more of the portable computer readable storage media 526, read via the respective R/W drive or interface 514 and loaded into the respective computer readable storage media 508.

The generic computer system may also include a network adapter or interface 516, such as a TCP/IP adapter card or wireless communication adapter (such as a 4G wireless communication adapter using OFDMA technology) for connection to a network 528. Application programs 511, such as the plan optimization application 310 (FIG. 3), may be downloaded to the computing device from an external computer or external storage device via a network (for example, the Internet, a local area network or other wide area network or wireless network) and network adapter or interface 516. From the network adapter or interface 516, the programs may be loaded onto computer readable storage media 508. The network may comprise copper wires, optical fibers, wireless transmission, routers, firewalls, switches, gateway computers and/or edge servers.

The generic computer system may also include a display screen 520, a keyboard or keypad 522, and a computer mouse or touchpad 524. Device drivers 512 interface to display screen 520 for imaging, to keyboard or keypad 522, to computer mouse or touchpad 524, and/or to display screen 520 for pressure sensing of alphanumeric character entry and user selections. The device drivers 512, R/W drive or interface 514 and network adapter or interface 516 may comprise hardware and software (stored on computer readable storage media 508 and/or ROM 506).

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

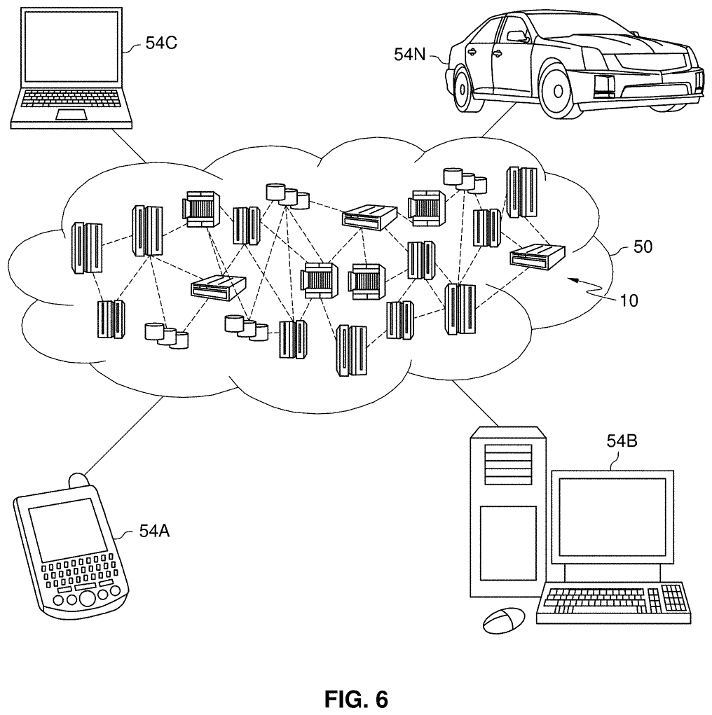

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

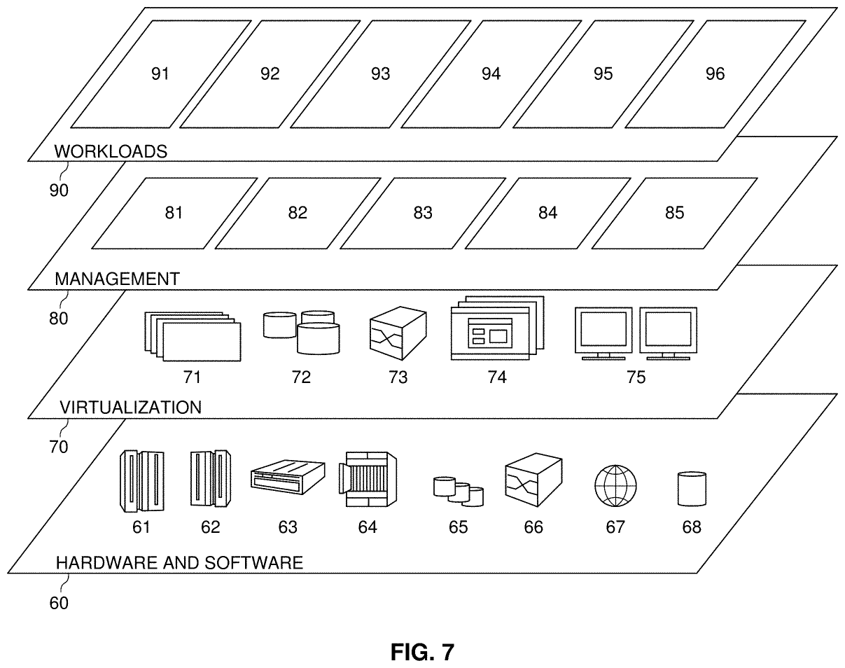

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and activity plan optimization 96.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While steps of the disclosed method and components of the disclosed systems and environments have been sequentially or serially identified using numbers and letters, such numbering or lettering is not an indication that such steps must be performed in the order recited, and is merely provided to facilitate clear referencing of the method's steps. Furthermore, steps of the method may be performed in parallel to perform their described functionality.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for developing an automated scheduling application, the method comprising:
receiving, by a computer, a user's schedule comprising a plurality of activities, wherein each activity in the plurality of activities is associated with one or more constraints;
associating, by the computer, biomedical parameters of the user with execution of the plurality of activities to determine an effect over time of each activity on the biomedical parameters, wherein the associating is performed using a first application programming interface;
based on the association of the biomedical parameters with the execution of the plurality of activities, identifying, by the computer, one or more evaluators comprising a predefined evaluation criteria to calculate a score for the user's schedule, wherein the determining is performed using a second application programming interface; and
performing, by the computer, a local search heuristic to modify and optimize the user's schedule, wherein the local search heuristic is performed using a third application programing interface.

2. The method of claim 1, wherein the one or more constraints are selected from the group consisting of: user availability, activity duration range, a temporal precedence among two or more activities, a relationship among two or more activities, an importance attribute associated with an activity, whether an activity is mandatory or optional, and a numerical or quantitative value associated with an activity.

3. The method of claim 1, wherein the biomedical parameters comprises a library of predefined biomedical parameters and user-defined biomedical parameters.

4. The method of claim 1, wherein the biomedical parameters are selected from the group consisting of: heart rate, blood pressure, blood sugar level, number of calories consumed, number of carbohydrates consumed, number of calories burned, and type and quantity of medication taken by the user.

5. The method of claim 1, wherein the predefined evaluation criteria comprises time spent outside a predefined safe zone for a given biomedical parameter, drug dosage consumed by the user, deviation from a scheduled activity, and deviation from a medical guideline.

6. The method of claim 1, wherein performing the local search heuristic to modify and optimize the user's schedule comprises:
generating a candidate schedule by modifying the user's schedule based on a set of allowed modifications, wherein the set of allowed modifications are determined based on the one or more constraints and the one or more evaluators,
calculating a score for the candidate schedule based on the one or more evaluators,
selecting the candidate schedule as a current schedule, based on the score of the candidate schedule being higher than the score of the user's schedule, and
generating a new user's schedule by restarting the local search heuristic based on a restart strategy selected from a set of restart strategies.

7. The method of claim 6, wherein the set of allowed modifications consist of at least one of a library of predefined allowed modifications and user-defined allowed modifications.

8. The method of claim 6, wherein the set of allowed modifications are selected from the group consisting of: changing a start time of an activity, changing a duration of an activity, adding an activity, removing an activity, changing a temporal ordering of two or more activities, applying a predefined time offset to one or more activities, and increasing or reducing a quantity associated with an activity.

9. The method of claim 6, wherein the set of restart strategies are selected from the group consisting of: restart from a previously-logged schedule, restart based on a user-defined schedule, restart based on a randomly-selected schedule, and restart based on a schedule obtained by abstracting problem aspects.

10. The method of claim 1, further comprising:
continuously monitoring an execution of the current schedule, wherein the monitoring is based on at least one of the execution of the plurality of activities and the biomedical parameters of the user.

11. The method of claim 10, further comprising:
based on the monitoring, calculating a new score for the current schedule.

12. A computer system for developing an automated scheduling application, the computer system comprising:
one or more processors, one or more computer-readable memories, one or more computer-readable tangible storage devices, and program instructions stored on at least one of the one or more storage devices for execution by at least one of the one or more processors via at least one of the one or more memories, wherein the computer system is capable of performing a method comprising:
receiving, by a computer, a user's schedule comprising a plurality of activities, wherein each activity in the plurality of activities is associated with one or more constraints;
associating, by the computer, biomedical parameters of the user with execution of the plurality of activities to determine an effect over time of each activity on the biomedical parameters, wherein the associating is performed using a first application programming interface;
based on the association of the biomedical parameters with the execution of the plurality of activities, identifying, by the computer, one or more evaluators comprising a predefined evaluation criteria to calculate a score for the user's schedule, wherein the determining is performed using a second application programming interface; and
performing, by the computer, a local search heuristic to modify and optimize the user's schedule, wherein the local search heuristic is performed using a third application programing interface.

13. The computer system of claim 12, wherein the one or more constraints are selected from the group consisting of: user availability, activity duration range, a temporal precedence among two or more activities, a relationship among two or more activities, an importance attribute associated with an activity, whether an activity is mandatory or optional, and a numerical or quantitative value associated with an activity.

14. The computer system of claim 12, wherein the biomedical parameters comprises a library of predefined biomedical parameters and user-defined biomedical parameters.

15. The computer system of claim 12, wherein the biomedical parameters are selected from the group consisting of: heart rate, blood pressure, blood sugar level, number of calories consumed, number of carbohydrates consumed, number of calories burned, and type and quantity of medication taken by the user.

16. The computer system of claim 12, wherein the predefined evaluation criteria comprises time spent outside a predefined safe zone for a given biomedical parameter, drug dosage consumed by the user, deviation from a scheduled activity, and deviation from a medical guideline.

17. The computer system of claim 12, wherein performing the local search heuristic to modify and optimize the user's schedule comprises:
generating a candidate schedule by modifying the user's schedule based on a set of allowed modifications, wherein the set of allowed modifications are determined based on the one or more constraints and the one or more evaluators,
calculating a score for the candidate schedule based on the one or more evaluators, selecting the candidate schedule as a current schedule, based on the score of the candidate schedule being higher than the score of the user's schedule, and
generating a new user's schedule by restarting the local search heuristic based on a restart strategy selected from a set of restart strategies.

18. The computer system of claim 17, wherein the set of allowed modifications consist of at least one of a library of predefined allowed modifications and user-defined allowed modifications.

19. The computer system of claim 17, wherein the set of allowed modifications are selected from the group consisting of: changing a start time of an activity, changing a duration of an activity, adding an activity, removing an activity, changing a temporal ordering of two or more activities, applying a predefined time offset to one or more activities, and increasing or reducing a quantity associated with an activity.

20. The computer system of claim 17, wherein the set of restart strategies are selected from the group consisting of: restart from a previously-logged schedule, restart based on a user-defined schedule, restart based on a randomly-selected schedule, and restart based on a schedule obtained by abstracting problem aspects.
